(19) United States
(12) Patent Application Publication
(10) Pub. No.: US 2009/0003522 A1
(43) Pub. Date: Jan. 1, 2009

(54) Method for radiation therapy delivery at varying source to target distances

(76) Inventors: Stanley Chien, Zionsville, IN (US); Lech Stanislaw Papiez, Roulett, TX (US); Robert Dale Timmerman, Westlake, TX (US)

Correspondence Address: Stanley Chien, 6729 E. Stonegate Dr., Zionsville, IN, 46077, US

(21) Appl. No.: 11/823,495

(22) Filed: Jun. 29, 2007

(51) Int. Cl. A61N 5/10

(52) U.S. Cl. 378/65

(57) ABSTRACT

A method for providing radiation therapy to target tissue in a patient provides for adjustment of the vertical position of the patient couch to account for errors introduced by the weight supported by the couch at its desired positions for treatment. The method further contemplates determining the initial location of the center of the target tissue with respect to the immobilization frame supporting the patient, to eliminate errors introduced by collateral position sensing equipment. The method is particularly suited for extended distance treatments wherein, in one embodiment, a tare is established based on the actual isocenter of the gantry and then a subsequent adjustment of couch position is made with respect to movement of the couch to position the target tissue at the virtual isocenter.

### REFERENCE TO RELATED APPLICATIONS

This application claims priority to co-pending provisional application No. 60/647,893, entitled “Method for Radiation Therapy Delivery at Varying Source to Target Distances”, filed on Jan. 28, 2005, the disclosure of which is incorporated herein by reference, and to co-pending provisional application No. 60/647,920, entitled “Relocatable Stereotactic Immobilization Apparatus”, filed on Jan. 28, 2005, the disclosure of which is incorporated herein by reference. This application also claims priority to international provisional application No. PCT/US2006/002883, entitled “Method for Radiation Therapy Delivery at Varying Source to Target Distances”, filed on Jan. 27, 2006, the disclosure of which is incorporated herein by reference, and to international provisional application No. PCT/US2006/002912, entitled “Relocatable Stereotactic Immobilization Apparatus”, filed on Jan. 27, 2006, the disclosure of which is incorporated herein by reference.

### BACKGROUND OF THE INVENTION

The present invention relates to a method for delivering radiation to a target at variable source-to-target distances. In other terms, the invention relates to a method for delivering radiation to a virtual isocenter. The invention has particular application to treatments for patients of larger girth.

Patient positioning systems are used for accurate and reproducible positioning of a patient for radiation therapy, diagnostic imaging, and certain surgical procedures. Immobilization devices support the patient and facilitate precise and accurate guidance for stereotactic interventions for defined three-dimensional target tissue within the patient's body, including the neck, chest, abdomen, pelvis and proximal thighs.

In a typical radiotherapy procedure, a gantry G (FIG. 1) directs a radiation beam at a machine isocenter 1. The gantry G rotates about a horizontal axis so that the radiation beam is always directed at the machine isocenter. The machine isocenter I is marked by the intersection of laser beams generated by several wall-mounted laser devices L in the treatment room (FIG. 2). The patient is supported on a motorized couch or table T, as shown in FIG. 2, that can be moved in the space surrounding and including the isocenter I so that the target tissue can be centered at the isocenter I. The gantry rotates about a horizontal axis by angles ranging from 0° to 360°. Thus, the treatment couch is configured to extend cantilevered beyond its base so that the gantry can rotate underneath the couch. The base of the couch includes mechanisms that accomplish this extension of the patient supporting couch. Moreover, in some installations the mechanisms also permit rotation of the couch in a horizontal plane so that the radiation beam can strike the target tissue at varying angles to achieve close to spherical application of radiation beams.

The need for effective patient immobilization for radiation therapy is well documented. Immobilization reduces normal tissue complication rates and allows increased irradiation of the target tissue. Historically, skin marks have been used to aid in target localization and repositioning. However, skin marks may migrate in successive treatments and the markings can shift with respect to underlying deeper target tissues. As a consequence, fiducial markings have been placed on patient immobilization frames, since these markings do not smear, fade or migrate. In some procedures, fiducial markings may be matched to skin markings to properly locate and position the target tissue relative to the isocenter.

To achieve comfortable immobilization, stereotactic body frames have been developed that support the patient on the couch or table T. One such frame F, depicted in FIG. 3, is disclosed in the provisional application No. 60/647,920 referenced and incorporated by reference above. This frame is specially configured to accept larger patients, and particularly patients with a girth that renders them unable to fit in current immobilization frames. While details of this frame are left to the above-referenced utility application, certain aspects will be discussed herein as they pertain to the present invention.

All accelerators are built so that the source of radiation can be rotated around the isocenter that is fixed in space. As explained above, in the typical procedure, the target tissue of the patient is positioned at the isocenter so the tissue can be irradiated from all directions as the accelerator gantry (such as gantry G in FIG. 1) rotates around the patient. This set-up is advantageous since the guiding lasers (lasers L in FIG. 2) and the field light of the linear accelerator can be used to set up the position of the patient's body accurately to properly relate the target tissue to the irradiating beams. This source-to-axis distance (SAD) approach typically does not require translational motions of the patient table.

In a typical installation, the position of the radiation source is constrained to a vertical plane and is located at 100 cm from the machine isocenter I. For machines that contain a multi-leaf collimator apparatus, the distance between the machine head and the machine isocenter I may be less than 43 cm. For some large-sized patients and for treatment techniques that require rotation of the patient couch (i.e., non-coplanar, stereotactic body radiation therapy (SBRT)), this distance is not sufficient because of the risk of collisions between the gantry head and either the patient, the couch, or treatment accessories.

This problem has been partially addressed by modified techniques called “extended distance” treatments or “extended distance source-to-axis distance” (EDSAD) treatments that allow a chosen constant distance D between the source and the target tissue, where D is greater than 100 cm (e.g., 120 cm). With these techniques, the center of the target tissue is positioned on the path of beams but not at the machine isocenter I. In order to irradiate the target tissue from different angles using the existing linear accelerators, EDSAD treatment can only be realized through movement of the target tissue center to various points on the surface of a sphere of radius D-100 cm centered at the isocenter of the machine. This set of points on the circle is referred to as a “virtual isocenter” (VIC). From the point of view of the target center of reference, this treatment is also iso-centric, with different radiation beams from the source on the gantry traveling to various points on the sphere of radius D-100 centered on the target tissue center. In other words, the virtual isocenter treatment establishes that for given gantry and patient couch angles, the central ray of the beam starts from the source, passes through the machine's isocenter at 100 cm from the source, and intersects the sphere of radius D-100 cm centered at the isocenter of the machine.

It can be appreciated that in a typical EDSAD treatment, the patient couch will necessarily be moved for each subsequent gantry and couch angle to maintain the center of the target tissue at a point on the virtual isocenter (VIC sphere), since the gantry is constrained to rotate about a fixed horizontal axis passing through the machine isocenter I. Thus, when the gantry is at its 0° position directly overhead the target tissue, the couch T must be lowered so that the tissue center is at a point on the VIC sphere. Likewise, when the gantry is at its 180° position, the couch must be raised. When the gantry is at a 90° position, lateral to the couch, the couch T must be moved laterally away from the gantry to position the target tissue at the VIC sphere.

Certain problems exist with this treatment technique. For instance, there is no easy way to use the room lasers to accurately guide the patient's body to the desired treatment location at the distance D. Since the location of the target tissue cannot be determined a prior in the couch coordinate system, it is not practical to determine the couch translation and rotation during the treatment. Moreover, even if the patient can be moved to the desired location by using coordinate transformation method and couch translational movements, a further problem arises due to bending of the couch that naturally occurs when the couch is cantilevered from the base. It can be appreciated that any cantilevered structure deflects downward due to its own weight. Add to that the weight of a patient and support frame mounted on the couch and it can be seen that the amount of deflection becomes non-negligible. For target tissue of relatively small size these deflection errors can mean the difference between properly irradiating the entire target tissue mass or irradiating more surrounding healthy tissue than target itself. When the couch and patient position is fixed during treatment, the target tissue center can be deliberately placed at the machine isocenter so any couch deflection is immaterial. However, in treatments requiring movement of the couch, couch deflection must be addressed at every new position of the couch. These practical problems make precise positioning of patients at extended distance EDSAD treatments wearisome. As a result, the available geometrical freedom of positioning the patient for optimal radiation exposure is significantly restricted in certain radiation therapies, particularly for SBRT treatments.

The present invention provides a novel approach to treatments using conventional accelerators that eliminates the need for laser guidance of the patient's body to a desired location at the extended treatment distance D. The present invention also eliminates the positioning inaccuracies of the target tissue relative to the beam that may be caused by deformation or bending of the patient couch.

### DESCRIPTION OF THE FIGURES

FIG. 1 is a perspective view of a radiation treatment apparatus.

FIG. 2 is a perspective view of a patient couch or table for use with the treatment apparatus of FIG. 1

FIG. 3 is a perspective view of a patient immobilization frame in accordance with one embodiment of the present invention.

FIG. 4 is a graph representing the relationship between couch bending and the longitudinal position of the couch.

FIG. 5 is a schematic representation of an exemplary coordinate transformation process used for embodiments of the present invention.

FIG. 6 is a representation of the gantry and couch movements in relation to the coordinate systems implemented in the present invention.

### DESCRIPTION OF THE PREFERRED EMBODIMENTS

For the purposes of promoting an understanding of the principles of the invention, reference will now be made to the embodiments illustrated in the drawings and described in the following written specification. It is understood that no limitation to the scope of the invention is thereby intended. It is further understood that the present invention includes any alterations and modifications to the illustrated embodiments and includes further applications of the principles of the invention as would normally occur to one skilled in the art to which this invention pertains.

The present invention relates to a process of expanding the treatment capability of linear accelerators and ultimately reducing patient treatment time. The invention integrates the linear accelerator gantry and patient couch with patient immobilization device, wall mounted laser system, target tissue locator, and position planning software. The use of target tissue locating and imaging devices, such as portal imaging, optical cameras, fluoroscopy and CT scanning, can enhance the implementation of the invention.

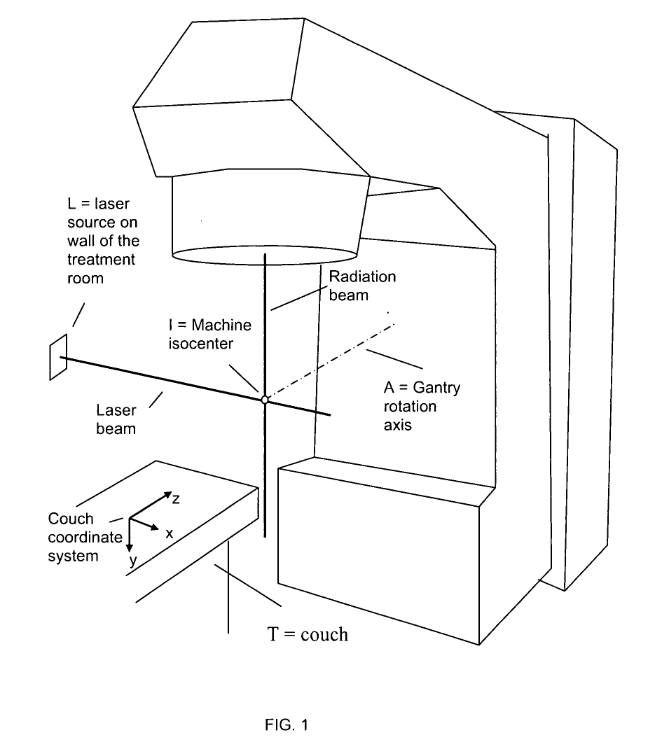

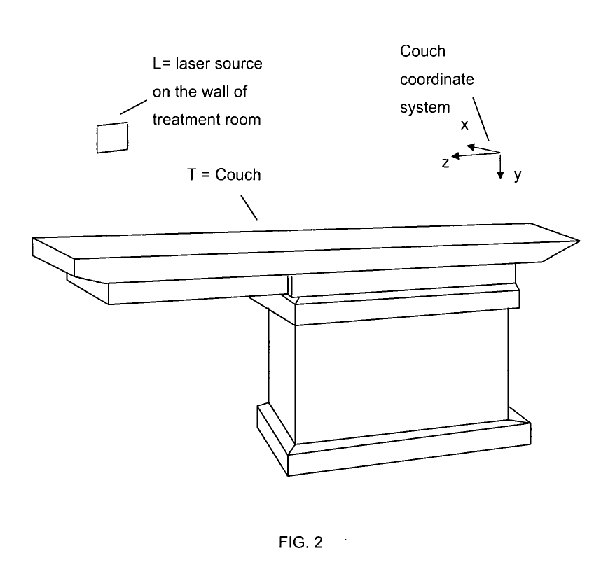

A typical therapeutic treatment system includes a gantry G that carries the linear accelerator. The gantry is configured to rotate in a single vertical plane about an isocenter I, as shown in FIG. 1, and around a patient couch or table T, as shown in FIG. 2. The gantry rotates through 360 degrees, and can be positioned at a specific angle with an accuracy of about one degree. The machine isocenter I can be located by the intersection of beams from the wall-mounted lasers L (FIG. 2) and by the intersection of the vertical plane containing the source of radiation with the axis of rotation of the gantry.

The patient couch T can also rotate about a vertical axis that passes through the isocenter of the gantry. The range of couch rotation is restricted to avoid collisions with the gantry. The table can be positioned at specific angle with an accuracy of about one degree. The couch may also provide translational motion in vertical, lateral and longitudinal directions (relative to the isocenter).

In accordance with the present invention, the patient couch establishes a three-dimensional (x,y,z) coordinate system (see FIG. 2) that is used to make patient measurements and to locate the center of the target tissue. For the purposes of reducing possible reading errors in operation, the table coordinate system is assigned so that numbers indicating the couch coordinates along all three axes are kept positive. Thus, in the lateral direction (x), the couch is at 0.0 (central position) when the longitudinal centerline of the couch (the z axis) passes through the isocenter. The lateral value of the couch movement increases from 0.0 cm as the couch moves to the right (relative to an observer facing the gantry), and decreases from 1000.0 cm when moving left. In a typical installation, the lateral range of movement is 974.0-1000.0 cm to the left, and 0.0-26.0 cm to the right. In the vertical direction, the 0.0 cm coordinate for downward movement in the couch scale is identified with coordinate 1000.0 cm for upward movement. The 0.0 cm (or 1000.0 cm) position corresponds to the intersection of the plane of the couch top surface with the isocenter. The range of couch vertical movement is 1000.0-960.0 cm upward and 0.0-66.0 cm downward. As reflected in FIG. 2, the origin of the couch coordinate system is offset from the isocenter along the z axis, so the couch moves longitudinally from 77.0 cm to 157.0 cm towards the gantry.

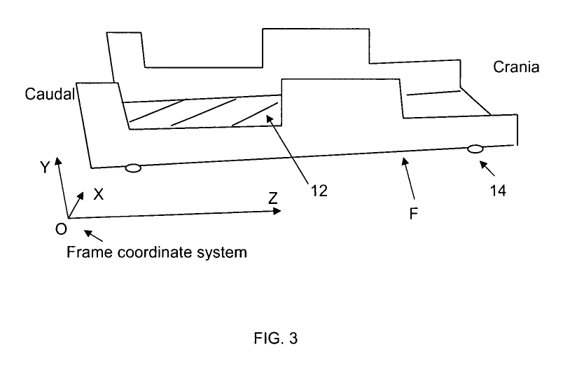

The present invention contemplates an immobilization frame F (FIG. 3) that is used to immobilize the patient during radiation therapy to ensure that the target tissue position does not shift in the frame during the procedure. In accordance with the present invention, the frame F defines its own three-dimensional coordinate system (X,Y,Z) as shown in FIG. 3 that can be mapped onto the table coordinate system (x, y, z) when the frame is locked to the couch T. The frame is preferably provided with a fixation mechanism 14 to fix the frame to the couch so that the frame cannot move independent of the couch. Details of certain embodiments of this fixation mechanism are disclosed in the above-mentioned provisional applications incorporated by reference.

The treatment room is preferably provided with a tissue locator to locate the target tissue within the patient. This can be achieved by imaging and reconstruction of the patient's three-dimensional anatomy. The images include CT data and may be supplemented by MRI or PET images. The three-dimensional images allow the treatment planner to determine the locations and sizes of the target tissue (e.g., tumors), as well as the sensitive strictures surrounding those targets. The three-dimensional images are related to fiducials 12 on the immobilization frame F, so that the target tissue positions are defined in the frame coordinate system. The position of fiducials is fixed relative to the origin of the frame coordinate system (OX, OY, OZ)

Planning software is used to determine the treatment angles in terms of the gantry and the couch directions and spatial movements. In accordance with the present invention, this software will evaluate the planned movements and determine whether collisions may result between the gantry and the patient couch or treatment accessories. The software will also warn the planner of treatment configurations that are unachievable at the time of irradiation.

Accurately positioning the patient in a desired position and orientation is the key for successful radiation therapy. In current treatment facilities, there are usually three measurement reference devices used to find the location of the patient relative to the system coordinates—namely, the wall-mounted laser beams, the field light of the linear accelerator, and readings from mechanical sensors associated with the rotating gantry and translating patient table. However, applicants have discovered that many sources of position errors arise when using the current techniques and position reference devices.

Looking first at the patient couch, the typical sensor readings for couch translation is in 1.0 mm (0.1 cm) increments, so that the maximum accuracy (E) of the couch readings in any translational direction is 0.5 mm. Therefore, the maximum combined inaccuracy (Ex²+Ey²+Ez²)^{0.5} is 0.87 mm. In the rotational degrees of freedom, both the gantry and the couch have one degree accuracy. The maximum accuracy for setting a target tissue at a specific location according to couch and gantry rotation values is thus limited by the accuracy of the couch and rotation measurements.

In experiments, disagreements may arise between the couch position measurements and the position data generated by the wall lasers. For instance, with the tissue positioned at a distance of 50 cm from the isocenter in one experiment, the measurement generated by projected laser beams was about 2.0 mm apart from the reading from couch position data. It is believed that this position discrepancy is caused by either rotation inaccuracy of the couch or inaccuracy of the laser installation. Further experiments showed that the error was primarily attributable to the laser installation. However, the couch motion in the present invention relies principally on the couch readings and not on position data generated by the wall lasers.

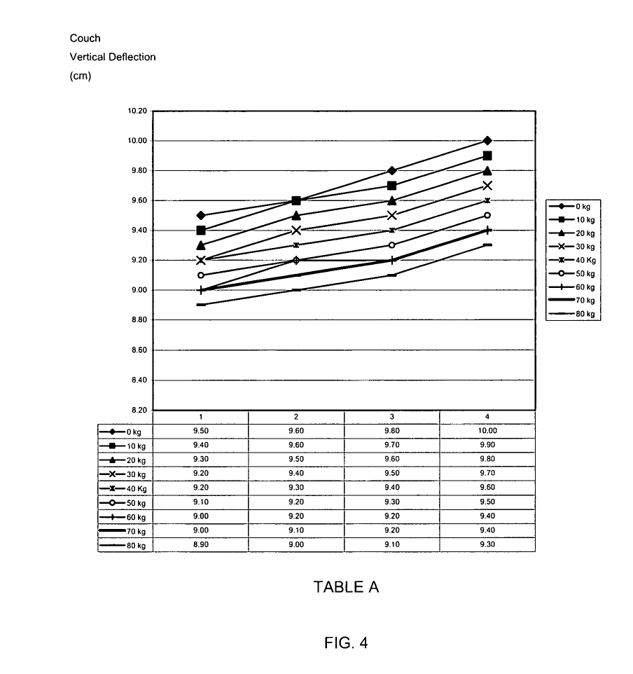

It was noticed that vertical position readings of the couch may also be inaccurate. The typical patient couch includes a bed that extends longitudinally toward the gantry relative to a support base. Thus, the bed is essentially cantilevered relative to the base. In most uses, the patient is situated on the couch bed with his/her head adjacent to the gantry. Thus, the cantilevered portion of the couch carries the majority of the patient's mass. To determine the effect of the patient weight to the vertical position reading error, we conducted experiments in that different cantilevered weights were supported on the couch extended to different longitudinal positions. The results of these experiments, summarized in the graph and Table A of FIG. 4, reveal that (1) when the couch is at z=78.8 cm, the couch vertical reading is 10 and there is no bending; (2) for every longitudinal position of the patient couch (column 1: z=156 cm; column 2: z=130 cm; column 3: z=112.8 cm; column 4: z=78.8 cm), adding weight increases the vertical downward deflection of the couch; and (3) as the couch extends (z increases) with the same weight, the bending increases (vertical value decreases).

For the purposes of the present invention, the absolute magnitude of the vertical deflections is not as important as the relative change between data cells in the table. For instance, the differences between vertically adjacent cells (i.e., constant longitudinal position with increasing weight) correspond to bending error due to changing weight. The differences between horizontally adjacent cells (i.e., constant weight with increasing longitudinal displacement) correspond to bending error introduced by the couch moving in the longitudinal direction.

A similar experiment was constructed to determine errors introduced by moving lateral movement of the couch. The following Table B summarizes the vertical displacement of the couch as a function of lateral displacement and cantilevered weight:

TABLE B

Lateral Displacement

| Weight | 980 cm | 990 cm | 0 cm | 10 cm | 20 cm |
|---|---|---|---|---|---|
| 80 Kg | 8.8 cm | 8.9 | 9.0 | 8.9 | 8.8 |
| 60 Kg | 8.9 | 9.0 | 9.1 | 9.0 | 8.9 |

As the above data reveals, couch movement and patient weight affect the vertical displacement of the table, and consequently causing the error in the couch position measurements. In accordance with one aspect of the present invention, this empirical data may be used to generate an error adjustment map due to bending of the couch as a function of longitudinal displacement, lateral displacement and equivalent weight applied to the couch. Preferably, the error adjustment map may be generated using curve-fitting methods to provide an optimized algorithm, equation or table look-up. It is contemplated that this error adjustment map will provide a couch vertical offset value as a function of input values for patient weight, longitudinal couch position and lateral couch position. It is further contemplated that this error adjustment map will be specific to each type of patient couch, due to mechanical differences between couches.

The experiment data shows that the couch vertical offset due to patient weight and couch position is cumulative, meaning that the vertical offset obtained from Table A for longitudinal movement is combined with the vertical offset from Table B for lateral movement. Thus, in one embodiment of the invention, two error adjustment maps are provided—one for longitudinal movement and one for lateral movement. These error adjustment maps are then used as described in more detail herein to accurately position the center of the target tissue at the virtual isocenter VIC.

The present invention contemplates a new process for radiation treatment using a combination of the couch, the immobilization frame and the linear accelerator.

Initiating the process requires the following input:
(a) the location of the origin of the immobilization frame in the couch coordinate system (Ox, Oy, Oz);
(b) the location of the center of the target tissue in the frame coordinate system (X,Y,Z); and
(c) the desired treatment set-up parameters (e.g., couch angle, gantry angle, and extended distance) at a particular virtual isocenter. It is contemplated that a predetermined series of parameters is provided to correspond to the total treatment protocol.

With respect to the first required input (the location of the frame origin), it is understood that any fixed point on an immobilization frame may be defined as its origin. To reduce the chance of reading mistakes, the origin of the frame coordinate system is situated as shown in FIG. 3 to ensure that all points to be referenced for treatment in the frame coordinates have positive values. The frame coordinate system is fixed relative to the patient couch T when the frame itself is locked to the couch, as described above. The frame is preferably locked to the couch at known locking positions on the couch.

In one approach to locate the frame origin, the couch is translated with the frame locked to the couch until the frame origin (Ox, Oy, Oz) in the couch coordinate system coincides with the gantry isocenter I. The translational couch movements, or move vector, necessary to move the frame origin to the isocenter will then correspond to the actual location of the frame origin relative to the couch origin. However, this approach is not always practical due to the range limitations of the couch movement. Another approach is to lock the frame to the couch and then drive the couch until a particular known reference point, such as a fiducial, in the frame coordinate system (x, y, z) is located at the isocenter. The couch position vector (Px, Py, Pz) in the couch coordinate system may be expressed as (Px, Py, Pz)=(Ox−x, Oy−y, Oz−z). Therefore, the origin of the frame coordinates (Ox, Oy, Oz) in the couch coordinate system can be determined as (Ox, Oy, Oz)=(Px+x, Py+y, Pz+z). This required input must be obtained for each type of the frame at each locking position on the couch, since the frame is at a different location on the couch for each different locking position.

The second input is the target tissue position in the frame coordinate system. With the patient in the immobilization frame F, an imaging scan, such as CT, MRI or PET, is performed. The target tissue, patient markers and fiducials 12 on the frame are ascertained in the images. The position of the target tissue in frame coordinates can be determined based on the relationship of the target tissue center to the fiducials, which have a known location relative to the frame origin and ultimately to the couch coordinate system, as determined in the prior step.

The final inputs are the desired treatment set-up parameters, such as couch angle, gantry angle and extended distance (especially important for large girth patients), from the treatment plan. The objective of the present invention is to easily and accurately determine the couch positions to satisfy the desired treatment, while avoiding or eliminating possible errors from the various sources described above. In other words, the objective is to achieve the couch and gantry movements necessary to follow the treatment plan without error and without collision between couch and gantry. Thus, these inputs need to be adjusted to correct various potential errors noted above.

One embodiment of the present invention contemplates a series of steps to accurately determine the couch positions during the treatment movements. These steps may include:
(1) determining the frame locking position to set the origin of the frame coordinates (Ox, Oy, Oz) in the couch coordinate system. In the manner described above;
(2) receiving the target tissue location in frame coordinates obtained as outlined above;
(3) partially correcting for couch bending error (called tare) while putting the target tissue at the machine isocenter and setting the gantry and couch at their zero angular positions;
(4) receiving the desired treatment angle for the couch and gantry and the treatment distance D;
(5) calculating the corresponding couch position at which the target tissue center is on the radiation beam at the desired distance D from the source, using coordinate transformation techniques; and
(6) further correcting the couch vertical error due to the motion of the couch in the longitudinal and lateral directions in accordance with the treatment protocol.

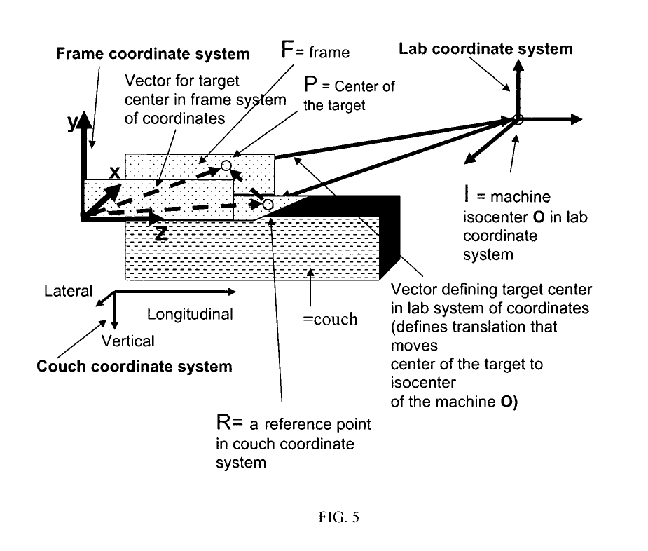

Steps (1), (2), and (4) are relatively straightforward and have been discussed above. The remaining steps require more detailed explanation and form an important part of the present invention. Step (3) entails partially correcting the couch vertical position error caused by the weight of the patient and the couch on the cantilevered portion of the couch. The effect of these cantilevered weights on the vertical couch coordinate can be measured and corrected using the following sub-steps of Step (3):
(3)(i) The patient is placed into the frame and the frame is locked to the couch according to the user inputs in Steps (1) and Step (2). The angles for both the gantry and the couch are set to zero and a coordinate transformation calculation is made for the lateral, vertical and longitudinal values (Cx, Cy, Cz) of the couch that corresponds to placing the center of the target tissue or tumor at the system isocenter. One form of this coordinate transformation is depicted in FIG. 5.
(3)(ii) Next, the couch is driven to move the target tissue to the position (Cx, Cy, Cz) calculated in sub-step (i). The actual target center location is checked for errors in reference to either, or a combination of, laser referenced measurements, port film, fluoral image, cone CT, etc. If there is no error (i.e., the actual location matches the calculated position), the center of the target tumor is presumed to be within the minimal expected error, or more particularly within a 0.87 mm radius of the system isocenter.
(3)(iii) Preferably, but optionally, the field light of the linear accelerator or the site laser may be used to verify the location of the fiducial marks of the frame. If the distance error in each direction is only a few millimeters, it is likely that these errors are just caused by the patient's weight. However, a large discrepancy is an indicator that there are other errors in the movement, measurement or calculation that need to be traced back and corrected before the process can be continued.
(3)(iv) As explained above with reference to Tables A and B, position errors will result when the couch is moved. These errors are due to patient, frame and couch weight and/or to mechanical inaccuracies inherent in the couch movement. In accordance with the present embodiment, this Sub-step (3)(iv) contemplates eliminating these errors by moving the couch to a “tare” position (Cx′, Cy′, Cz′) in order to re-position the tumor at the isocenter. By doing tare in the lateral and longitudinal direction, errors introduced by mechanical inaccuracy in the couch movement mechanism and by the frame locking mechanism can be removed. By doing tare in the vertical direction, the bending caused by the weight of the couch and the patient can be compensated. The amount of offset necessary to move the couch to the tare position in the vertical direction may be obtained from the error adjustment maps discussed above.
(3)(v) The origin of the frame is then adjusted from original position (Ox, Oy, Oz) to the tare position (Ox′, Oy′, Oz′) in the couch coordinate system. This adjustment of the frame origin in effect compensates for the errors described in Sub-step (3)(iv). Thus, a coordinate transformation Ox′=Ox+(Cx−Cx′), Oy′=Oy+(Cy−Cy′), and Oz′=Oz+(Cz′−Cz), may be employed to find the revised couch position (Cx′, Cy′, Cz′) that can be substituted for the position (Cx, Cy, Cz) used in the prior sub-steps.

The sub-steps of Step (3) can correct the couch bending error caused by the weight on the couch and caused by the longitudinal position of the couch. The end result of the sub-steps of Step (3) is that accounts for tare by shifting the coordinate system origin from which subsequent position determinations are made. In other words, a revised couch position is provided for use in the subsequent Steps (4)-(6).

Step (5) attempts to correct the error introduced by moving the tissue center from the machine isocenter to the virtual isocenter used in the EDSAD treatment protocol described above. Known coordinate transformation techniques may be used to calculate the destination couch values (Cdx, Cdy, Cdz) according to the desired gantry and couch angles obtained in Step (4) and using the adjusted frame origin (Ox′, Oy′, Oz′) obtained in Step (3). Since the transformation equations depend on how the coordinate systems are defined, the sequence of steps defining a preferred coordinate transformation protocol for the coordinate systems described herein is as follows:
(5)(i) It is assumed that the origin of the global coordinate system for the treatment facility is at the machine isocenter I. Since the desired gantry angle Θ and distance D between the virtual isocenter and the radiation source are known from user input, the location of the virtual isocenter (VIC) in the global coordinate system can be found.
(5)(ii) Since the desired couch angle Φ and the relationship between the couch coordinate system (x, y, z) and the global coordinate system are known, the VIC coordinates in the couch coordinate system that corresponds to VIC coordinates in the global coordinate system can be found.
(5)(iii) Since the tumor center position in the frame coordinate system is known from user input, the manner in which the couch must be driven to move the center of the tumor from (Ox′, Oy′, Oz′) to VICc=(VICcx, VICcy, VICcz) can be calculated.

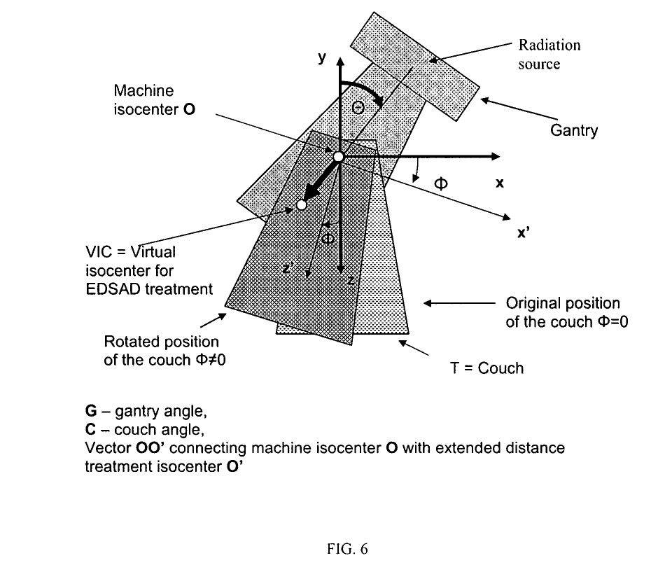

Thus, Step (5) provides a couch movement value that can position the tumor at the virtual isocenter for any desired gantry angle and couch angle. These coordinate transformations are represented in FIG. 6. In this figure, the gantry G is depicted rotating through an angle Θ in a vertical plane about a longitudinal axis y passing through the isocenter I. The patient couch or table T has its surface in the plane defined by the x and y axes and is depicted rotating about the vertical axis z centered at the isocenter I through the angle Φ. In an EDSAD treatment, it can be appreciated that the vector from the machine isocenter I to the virtual isocenter VIC will be aligned with the line between the radiation source and the isocenter. The location of the virtual isocenter VIC in the global coordinates is given by (−Dsin Θ, Dcos Θ, 0), which is referred to as the vector OO′ in FIG. 6.

In accordance with the steps outlined above, the couch movements for the treatment protocol are observed in the couch coordinate system. Thus, a vector transformation is necessary into couch coordinates. This transformation for a movement vector V is given by the following:

$$V = [ \begin{matrix}cos Φ & 0 & - sin Φ \\ 0 & 1 & 0 \\ sin Φ & 0 & cos Φ\end{matrix} ] [ \begin{matrix}- D \cdot sin Θ \\ D \cdot cos Θ \\ 0\end{matrix} ] = [ \begin{matrix}- D \cdot sin Θ cos Φ \\ D \cdot cos Θ \\ - D \cdot sin Θ cos Φ\end{matrix} ]$$

where D is the extended distance value, Θ is the gantry rotation angle and Φ is the couch rotation angle. Application of this vector transformation places the center of the target tissue at the point O′ or the VIC for the extended distance treatment.

However, due to the motion of the tumor center from machine isocenter to the virtual isocenter, a new vertical couch bending error is introduced. Thus, the final Step (6) of this process is to correct this vertical bending error. Note that the vertical bending error at the isocenter was initially corrected in Step (3), but since the tissue center has been moved from system isocenter I (or origin O in FIG. 6) to the virtual isocenter position VIC (or origin O′ in FIG. 6), an additional correction is required. It can be remembered that the movement to the virtual isocenter is part of the extended treatment procedure that relies upon positioning the patient couch or table at various locations in a sphere at the virtual isocenter. Thus, the additional error arises when the couch is moved from tare point (Ox′, Oy′, Oz′) to the virtual isocenter VICC. The maximum value of this error can be estimated as follows. If the desired distance from the source of the radiation beam and the target tissue is D (where D=120 cm is adequate for almost all sized patients for a typical linear accelerator), then the distance between (Ox′, Oy′, Oz′) and (VICcx, ViCcy, VICcz) is (D-100) using the 100 cm distance from radiation source to the machine isocenter I. (Note that the Ox′, Oy′, Oz′ values represent the origin at the tare point obtained in Step (3)). Thus, the relationship below follows:

(D−100)²=(Ox′−VICCx)²+(Oy′−VICcy)²+(Oz′−VICcz)²

The couch can move at most (D-100) centimeters in any direction from the tare point. When the tare is performed at (Ox′, Oy′, Oz′), all errors are eliminated. However, when the couch is actually moved to VICc according to the calculated transformation value, the target tissue center does not remain at the virtual isocenter, as mathematically predicted, due to changes caused by the weight longitudinal and lateral error relationships expressed in Tables A and B. In order to obtain the true couch movement vector it is first necessary to correct the error caused by the longitudinal motion of the couch. The error adjustment maps discussed above may be consulted to estimate the vertical error caused by the longitudinal movement of the couch VICcz-Oz′ and ultimately to generate a corrected virtual isocenter value of VICcy′.

Next the vertical error due to the lateral motion of the couch is corrected. Again, the error adjustment maps discussed above can be used to estimate the error introduced by the lateral change VICcx-Ox′, and to generate an adjusted coordinate ViCcy′. The same process is instituted to correct for the weight of the couch and patient, again using the error adjustment maps.

All of the steps of this preferred embodiment of the invention can be implemented in software. The system operator need only input the required information set forth above and establish the tare for the patient. The software then performs the coordinate transformations described above to transform the operator input couch and gantry moves to couch lateral, vertical and longitudinal moves that compensate for the errors described herein. The calculations necessary to determine the adjusted couch move protocol may be done immediately when the treatment protocol data is entered and before the treatment is commenced. Thus, if an operator enterers a sequence of desired gantry and couch rotations, software implementing the present invention will determine the appropriate couch movements necessary to maintain the center of the target tissue at the machine isocenter I for standard coplanar treatments or at the virtual isocenter for non-coplanar extended distance treatments. This software may communicate the appropriate couch position data to a control system in the base of the patient couch or table T that is responsible for mechanically moving the couch.

It is contemplated that error adjustment maps for correction of lateral and longitudinal movement errors are maintained in a database accessible by the software. The data populating these maps is preferably specific to each patient table T and may be generated by the couch manufacturer or empirically by tests at the treatment facility.

It is further contemplated that the software implementing the steps of the present invention will work through a user interface to permit entry of the Inputs (a)-(c) described above. Thus, the operator can enter the location of the immobilization frame in couch coordinates (Input (a)) and the target tissue center position in frame coordinates (Input (b)) following the procedures outlined above. The Inputs (c) are independent of the couch and its movement errors and are instead determined by the desired radiation treatment protocol.

The present invention contemplates a method that enables fast and precise positioning of a target tissue at varying source to target distances, or at a virtual isocenter. According to the present invention, a tare is obtained at the system isocenter (in global coordinates), so that the bending error introduced by the effective weight of the patient at this point is already corrected. When the couch is moved in lateral and longitudinal directions, the effective weight of the patient that causes couch or table bending also changes. This change of effective weight is difficult to quantify accurately; however for the purposes of the present invention this change can be estimated. This estimated change due to effective weight can be used for adjusting the bending error.

It has been found that the bending error introduced by lateral and longitudinal movements can be treated separately since these errors are generally independent based on empirical data. This independence means that bending errors can be isolated into a bending error maps as a function of weight only, longitudinal position only, and lateral couch position only.

While the invention has been illustrated and described in detail in the drawings and foregoing description, the same should be considered as illustrative and not restrictive in character. It is understood that only the preferred embodiments have been presented and that all changes, modifications and further applications that come within the spirit of the invention are desired to be protected.

What is claimed is:

1. A method for providing radiation therapy to target tissue within a patient supported on a couch movable in lateral, vertical and longitudinal directions relative to a rotating gantry operable to generate a radiation beam passing through an isocenter, the method comprising:
identifying a three-dimensional frame coordinate system relative to an immobilization frame;
positioning the patient within the immobilization frame;
determining the location of the target tissue relative to the frame coordinate system;
fixing an immobilization frame to the couch so that it moves with the couch;
determining a vector between the target tissue location and the isocenter;
using the vector to direct movement of the couch so that the target tissue location is coincident with the isocenter; and
then applying radiation to the target tissue.

2. The method for providing radiation therapy of claim 1, wherein determining a vector includes:
identifying a three-dimensional couch coordinate system relative to the couch;
determining the location of the origin of the frame coordinate system in the couch coordinate system;
combining a vector corresponding to the location of the target tissue in frame coordinates with a vector corresponding to the location of the frame system origin in couch coordinates to obtain the target tissue location in couch coordinates; and
then determining the vector between the target tissue location and the isocenter in couch coordinates.

3. The method for providing radiation therapy of claim 1 further comprising before applying the radiation;
obtaining a tare with the target tissue location at the isocenter;
receiving operator inputs for desired gantry and couch angles during the treatment; and
using the tare with the operator inputs to determine a corrected couch position.

4. The method for providing radiation therapy of claim 3, wherein obtaining a tare includes:
determining an adjusted frame origin location to account for errors in the position of the target tissue relative to the isocenter; and
using the adjusted frame origin location to adjust the target tissue location when determining the vector between the target tissue location and the isocenter.

5. The method for providing radiation therapy of claim 4, wherein:
the errors are errors in vertical position of the couch as a function of the weight supported by the couch and one or both of the lateral or longitudinal positions of the couch; and
an error adjustment map is provided with vertical adjustments to the couch position to compensate for such errors when determining the vector between the target tissue location and the isocenter.

6. A method for providing radiation therapy to target tissue within a patient supported on a couch movable in lateral, vertical and longitudinal directions relative to a rotating gantry operable to generate a radiation beam passing through an isocenter, the method comprising:
receiving operator inputs for movement of the couch to desired couch angles at which radiation is applied to the target tissue;
adjusting the vertical position of the couch as a function of the weight supported by the couch when the couch is moved to the desired couch position;
moving the couch with the patient supported thereon to the adjusted desired couch position; and
then applying radiation to the target tissue.

7. The method for providing radiation therapy of claim 6, wherein adjusting the vertical position includes:
providing an error adjustment map of vertical adjustments as a function of the weight supported by the couch and one or both of the lateral or longitudinal positions of the couch;
obtaining a vertical adjustment from the error adjustment map based on the actual weight of the patient; and
adjusting the operator input by the vertical adjustment.

8. The method for providing radiation therapy of claim 6, further comprising:
positioning the patient on the couch;
determining the target tissue location;
moving the couch longitudinally toward the gantry so that the target tissue location is at the virtual isocenter;
determining an initial vertical adjustment to correct bending of the couch when the target tissue location is at the isocenter; and
combining the initial vertical adjustment with the operator inputs to generate new desired couch positions.

9. The method for providing radiation therapy of claim 8, in which the therapy is extended distance treatment at a distance D from the gantry to produce a virtual isocenter, wherein:
the initial vertical adjustment is determined with the target tissue location at the isocenter; and
the desired couch positions and new couch positions are determined to orient the target tissue location at the virtual isocenter.
